(19) United States
(12) Patent Application Publication
(10) Pub. No.: US 2024/0416023 A1
(43) Pub. Date: Dec. 19, 2024

(54) MEDICAL SYSTEMS, DEVICES, AND RELATED METHODS FOR WOUND THERAPY

(71) Applicant: Boston Scientific Scimed, Inc., Maple Grove, MN (US)

(72) Inventors: Matthew MONTAGUE, Galway (IE); Louis McNERN, County Donegal (IE)

(73) Assignee: Boston Scientific Scimed, Inc., Maple Grove, MN (US)

(21) Appl. No.: 18/741,316

(22) Filed: Jun. 12, 2024

Related U.S. Application Data
(60) Provisional application No. 63/508,020, filed on Jun. 14, 2023.

(51) Int. Cl. A61M 1/00; A61B 1/00

(52) U.S. Cl. CPC A61M 1/915; A61B 1/00094; A61M 2205/0266; A61M 2205/32; A61M 2205/6081

(57) ABSTRACT

A medical device includes a wire or tube, and a porous body coupled a distal portion of the wire or tube. The wire or tube is configured to be transitioned from a first configuration to a second configuration. In the first configuration, the wire or tube is in a flat or straight configuration. In the second configuration, the wire or tube is expanded to a curved or helical shape.

### CROSS-REFERENCE TO RELATED APPLICATIONS

This application claims the benefit of priority to U.S. Provisional Application No. 63/508,020, filed on Jun. 14, 2023, which is incorporated by reference herein in its entirety.

### TECHNICAL FIELD

Various aspects of the disclosure generally relate to medical systems, devices, and related methods that may be used to treat a subject. In particular, aspects of the disclosure relate to medical systems, devices, and methods for wound therapy, such as endoscopic vacuum therapy that includes applying negative air pressure to tissue for wound therapy.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Limited treatment options exist for managing such wounds, which have significant morbidity and mortality rates. Options include surgical re-operation and endoscopic placement of a stent or one or more clips. Surgery is invasive and also has high morbidity and mortality rates. Endoscopic stent placement is a less invasive option. The placed stent, however, can migrate from the intended location and/or wall off an infection at the treatment site, which may exacerbate the infection and/or inhibit drainage.

The systems, devices, and methods of the current disclosure may rectify one or more of the deficiencies described above or address other aspects of the art.

### SUMMARY

Each of the aspects disclosed herein may include one or more of the features described in connection with any of the other disclosed aspects.

Aspects of the disclosure relate to, among other things, systems, devices, and methods for treating a subject. Aspects of this relate to medical systems, devices, and methods for wound therapy, such as endoscopic vacuum therapy that includes applying negative air pressure to tissue for wound therapy.

In some aspects, a medical device may include a wire or tube, and a porous body coupled a distal portion of the wire or tube. The wire or tube may be configured to be transitioned from a first configuration to a second configuration. In the first configuration, the wire or tube may be in a flat or straight configuration. In the second configuration, the wire or tube may be expanded to a curved or helical shape.

The medical device may include one or more of the following aspects. At least a distal portion of the wire or tube may include or may be formed of a shape memory material. The wire or tube may be a vacuum tube configured to be coupled to a negative pressure source. The vacuum tube may be cylindrical with a central lumen extending along a central longitudinal axis of the vacuum tube. The vacuum tube may include a plurality of ports disposed along a length of a distal portion of the vacuum tube. The plurality of ports may be configured to apply negative pressure provided by the negative pressure source.

In the second configuration, the curved or helical shape of the wire or tube may cause the porous body to have a helical shape. The porous body may be a sponge. The porous body may be cylindrical. The wire or tube may be configured to be deployed into a wound cavity, and a length of wire vacuum tube within the wound cavity may be configured to be reduced as the wound cavity reduces in size.

The wire or tube may remain in the first configuration when exposed to a first temperature, and may transition to the second configuration upon being exposed to a second temperature. The second temperature may be body temperature. The porous body may cover only a portion of a distal end of the wire or tube. The porous body may cover an entirety of a distal end of the wire or tube. The medical device may include a plurality of markers disposed along a length of a portion of the wire or tube. The plurality of markers may be radiopaque.

In one or more other aspects, a medical device may include a vacuum tube configured to be inserted into a working channel of an endoscope. The vacuum tube may be configured to be coupled to a negative pressure source. The medical device may also include a porous body coupled a distal portion of the vacuum tube. The vacuum tube may be configured to be transitioned from a first configuration to a second configuration. In the first configuration, the vacuum tube may be flat or straight. In the second configuration, the vacuum tube may be expanded and distal portions of the vacuum tube and the porous body may have a helical shape.

The medical device may include one or more of the following aspects. The vacuum tube may remain in the first configuration when exposed to a first temperature, and may transition to the second configuration upon being exposed to a second temperature. The medical device may also include a plurality of markers disposed along a length of the vacuum tube. The plurality of markers may be radiopaque and/or color coded.

In one or more additional aspects, a medical device may include a wire or tube. At least a distal portion of the wire or tube may include or may be formed of a shape memory material. The wire or tube may include a plurality of radiopaque markers. A porous body may be coupled to the distal portion of the wire or tube. The wire or tube may be configured to be transitioned from a first configuration to a second configuration. In the first configuration, the wire or tube may be in a flat or straight configuration. In the second configuration, the wire or tube may be expanded to a curved or helical shape.

The medical device may include one or more of the following aspects. The wire or tube may be a vacuum tube configured to be coupled to a negative pressure source. The vacuum tube may include a central lumen extending along a central longitudinal axis of the vacuum tube. The vacuum tube may include a plurality of ports disposed along a length of a distal portion of the vacuum tube. The plurality of ports may be configured to apply negative pressure provided by the negative pressure source.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “distal” refers to a direction away from a user/toward a treatment site, and the term “proximal” refers to a direction toward a user. The drawings may include arrows labeled “P” and “D,” indicating proximal and distal directions, respectively. The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows a perspective view of an exemplary medical system, according to some embodiments.

FIG. 2 shows a side view of a distal portion of an exemplary medical device, according to some embodiments.

FIG. 3 shows a perspective view of an exemplary medical device, according to some embodiments.

FIG. 4A shows a perspective view of an exemplary medical device, according to some embodiments.

FIG. 4B shows a side enlarged view of a portion of the medical device of FIG. 4A.

### DETAILED DESCRIPTION

Endoluminal vacuum therapy (EVAC) is an adaptation of negative pressure wound therapy (i.e., vacuum therapy or wound vac), which may be used for external treatment of chronic, non-healing wounds, where a vacuum-sealed sponge is inserted into the wound and a negative pressure is applied to the sponge to promote drainage. In a typical EVAC procedure, negative pressure is delivered to a wound site internally within the GI tract, for example through a nasogastric tube having a sponge at its terminal end. The sponge may be placed endoscopically into a perforation, leak, or other wound, and negative pressure may then be applied to promote drainage from the wound.

There are presently no EVAC devices or kits indicated for use in the United States. As a result, surgeons typically create their own devices by attaching sponges taken from external wound vacuum kits to nasogastric tubes. Although these devices may be effective in draining a wound within the GI tract, the sponge may need be replaced throughout the healing process at regular intervals (approximately once every 2-3 days), with increasingly smaller sponges as the wound heals and decreases in size. In some instances, the sponge may need to be replaced up to 70 times. This need to continuously replace the sponges may result in patient discomfort, as the nasogastric tube is inserted and removed via the nasal cavity. Additionally, the risk for infection may be increased, for example, each time the sponge is changed.

Accordingly, embodiments of this disclosure include devices, systems, and methods specifically for EVAC procedures. In some embodiments, EVAC may include endoluminal placement of a porous body (e.g., a sponge or other like material) into a wound site, for example a perforation, cyst, a leak, or an anastomosis.

The porous body may be placed within a wound via a catheter, scope (endoscope, bronchoscope, colonoscope, etc.), tube, or sheath, which may be inserted into the GI tract via a natural orifice. The orifice may be, for example, the nose, mouth, or anus, and a distal end of the catheter, scope, tube, or sheath (and thus the porous body) may be positioned in any portion of the GI tract, including the esophagus, stomach, duodenum, large intestine, or small intestine.

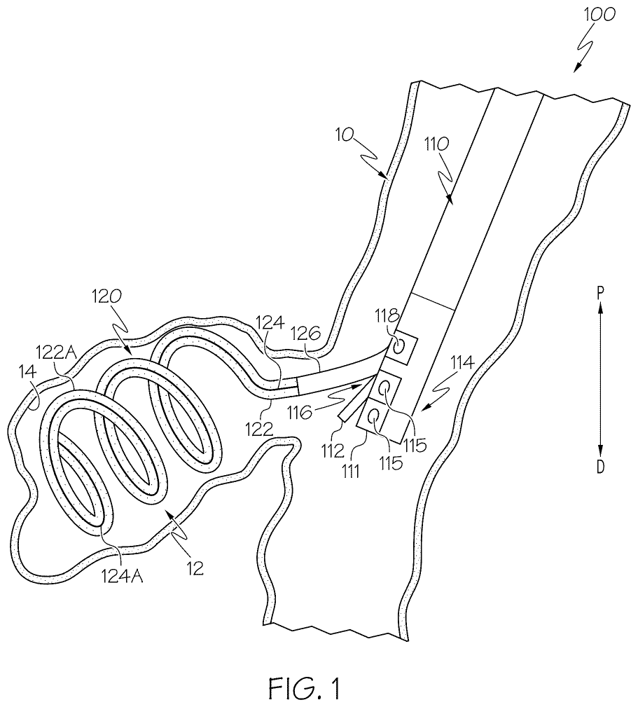

FIG. 1 depicts an exemplary medical system 100. Medical system 100 may include an insertion device or an endoscope 110, which may be inserted into an esophagus 10 of a patient. Endoscope 110 may include an umbilicus (not shown), which may connect a proximal portion of endoscope 110 to sources of, for example, air, water, suction, power, etc., as well as to image processing and/or viewing equipment. In some embodiments, endoscope 110 may additionally include at least one imaging element 115 and at least one lighting element 118 disposed at a distal end 114 of endoscope 110. Imaging element 115 and lighting element 118 may aid in accurately positioning endoscope 110 adjacent a wound during an EVAC procedure. For example, a user may utilize imaging element(s) 115 and lighting element 118 to aid in positioning distal end 114 of endoscope 110 adjacent to a wound cavity 12, for example, an anastomotic cavity. The user may then deploying or otherwise deliver a medical device 120 into wound cavity 12 (e.g., through a portion of endoscope 110). As discussed in detail below, medical device 120 includes a porous body 122 coupled to at least one wire 124 to perform therapy on wound cavity 12.

In some embodiments, medical device 120 may be inserted into a working channel of endoscope 110, which terminates in a working channel opening 116. Distal end 114 of endoscope 110 may also include an elevator 112, which may be disposed within a distal portion of the working channel, for example, adjacent to working channel opening 116. Elevator 112 may be movable between at least two configurations, and elevator 112 may be configured to change an orientation of medical device 120 when a portion of medical device 120 is positioned adjacent to and/or distal of elevator 112. Elevator 112 may alternatively be referred to as a swing stand, pivot stand, raising base, or any suitable other term. In some embodiments, elevator 112 may be pivotable via, e.g., an actuation wire or another control element coupled to and/or extending through endoscope 110. In some aspects, although not shown, endoscope 110 may include a handle with one or more actuators, for example, to control the movement of elevator 112, the activation of one or more imaging element(s) 115 and lighting element 118, a control a deflection, position, or orientation of distal end 114, and/or otherwise control one or more aspects of distal end 114 and its components.

It is noted that FIG. 1 illustrates distal end 114 of endoscope 110 as being “side-facing.” In a side-facing embodiment, imaging element(s) 115, working channel opening 116, and lighting element 118 may be disposed on a radially outer side of distal end 114, so that they point in a radially outward direction, approximately perpendicularly to a longitudinal axis of distal end 114 of endoscope 110. In other aspects, distal end 114 of endoscope may be “forward-facing.” In other words, features of distal end 114 (e.g., imaging element(s) 115, working channel opening 116, and lighting element 118) may face distally (i.e., forward of a distalmost face 111 of distal end 114). This disclosure also encompasses other configurations of distal end 114 as being “forward-facing.” Endoscope 110 may additionally include some components that are side-facing and other components that are forward-facing.

Although insertion device or endoscope 110 is discussed above as being an endoscope, this disclosure is not so limited. Although the disclosure may refer at different points to a an endoscope, it will be appreciated that, unless otherwise specified, duodenoscopes, endoscopes, gastroscopes, endoscopic ultrasonography (“EUS”) scopes, colonoscopes, ureteroscopes, bronchoscopes, laparoscopes, cytoscopes, aspiration scopes, sheaths, catheters, or any other suitable delivery device or insertion device may be used in connection with the systems, devices, elements, assemblies, methods, etc. described herein.

Still referring to FIG. 1, as mentioned above, medical device 120 may include porous body 122 coupled to wire 124. Medical device 120 may also include one or more tubes, for example, outer tube 126. Wire 124 and porous body 122 may be movable relative to the one or more tubes, for example, to outer tube 126. As discussed in detail below, extending wire 124 and porous body 122 from outer tube 126 may allow for one or more of wire 124 and/or porous body 122 to transition from a first configuration to a second configuration. Furthermore, in some aspects, a proximal portion of outer tube 126 may be coupled to a suction source, such that negative pressure may be applied to wound cavity 12 through outer tube 126. In some aspects, fluid within wound cavity 12 may flow along wire 124 and/or porous body 122, for example, via capillary action, with or without negative pressure being applied through outer tube 126.

Porous body 122 may be cylindrical or ring-shaped, for example, with a generally circular opening in the middle of porous body 122. Porous body 122 may be coupled to wire 124 by being wrapped around a portion (e.g., a distal portion) of wire 124. In some embodiments, porous body 122 may be a sponge or otherwise at least partially absorbent. In some embodiments, porous body 122 may include any suitable biocompatible material that may absorb liquids and/or permit liquid to pass therethrough, for example, via negative pressure. The material may be flexible, compressible, porous, hydrophilic, sterile, and/or disposable. The porous body material may be an open-cell foam. Suitable materials may include polyurethanes, polymers with ester and/or ether functional groups, composite materials, and any other medical-grade material or materials.

In some embodiments, wire 124 may be generally cylindrical, for example, either being solid or including one or more hollow openings in a center of wire 124. Wire 124 may be formed from a shape memory material, for example, Nitinol. Alternatively, wire 124 may be formed from a polymeric material. In some embodiments, wire 124 may be a tube, for example, a nasogastric tube. Wire 124 may be configured to be heat set into a certain configuration prior to being inserted into the working channel. For example, wire 124 may be configured to maintain a first configuration (e.g., a straight and/or flat configuration) at a first temperature, and be transitioned to a second configuration (e.g., an expanded configuration) upon reaching a second temperature, for example, body temperature. Alternatively or additionally, as discussed below, wire 124 be in the first configuration when enclosed or sheathed by an outer tube, and wire 124 may transition from the first configuration to the second configuration when unenclosed, unsheathed, or extended from the outer tube.

As shown in FIG. 1, at least a distal portion 124A of wire 124 may have a curved or helical structure, for example, in the second (e.g., expanded) configuration. In some embodiments, because porous body 122 may be coupled to wire 124, a distal portion 122A of porous body 122 may mimic the helical structure of distal portion 124A of wire 124. Furthermore, porous body 122 may be coupled to wire 124, such that porous body 122 transitions between respective configurations with wire 124.

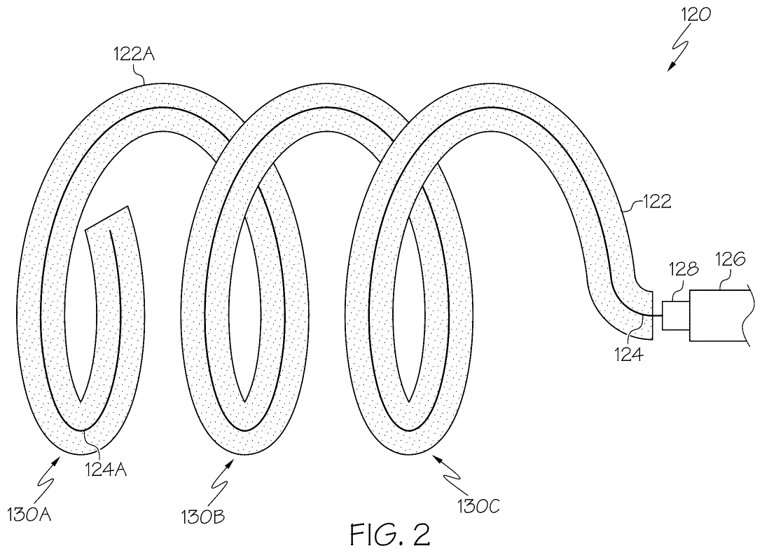

FIG. 2 is a side view of the distal portion of medical device 120 in the second or expanded configuration. As shown, porous body 122 is coupled to or otherwise surrounding wire 124. In the second or expanded configuration, wire 124 and porous body 122 may form three loops 130A, 130B, 130C, as shown. Alternatively, wire 124 and porous body 122 may form fewer or more loops. Furthermore, wire 124 may be movable (e.g., distally extendable and proximally retractable) relative to one or more tubes. As mentioned above, wire 124, and thus porous body 122, may be movable relative to outer tube 126. Moreover a proximal portion of wire 124 may be coupled to a sheath or an inner tube 128. In some aspects, inner tube 128 may be movable relative to outer tube 126, for example, to control a position of wire 124, and thus a configuration of wire 124 and porous body 122. Furthermore, in some aspects, wire 124 and/or inner tube 128 may be disconnectable or frangible, for example, such that wire 124 (with porous body 122) may be delivered (e.g., deployed into wound cavity 12). Wire 124 may then remain in wound cavity 12, and the remainder of medical device 120 and endoscope 110 may be removed from the patient (e.g., out of esophagus 10).

The curved or helical structure of distal portion 124A of wire 124 and distal portion 122A of porous body 122, in the second configuration, may provide several advantages. For example, the curved or helical structure may help to increase the surface area of porous body 122 in wound cavity 12, which may help to allow porous body 122 to collect (e.g., absorb) more fluid from wound cavity 12 than a typical cylindrical sponge. Additionally, the increased surface area and increased absorbency may help to allow for medical device 120 (e.g., including porous body 122) to remain within wound cavity 12 for a longer period of time (i.e., with fewer removals and/or replacements).

Furthermore, the curved or helical structure of porous body 122 and wire 124 may help to allow porous body 122 to more easily contact an inner wall 14 of wound cavity 12. As a result, as wound cavity 12 decreases in size throughout the healing process, inner wall 14 may abut and/or press against porous body 122, which may help push or urge the distal portion of medical device 120 out (e.g., proximally) of wound cavity 12. For example, at least a portion of wire 124 may be flexible, for example, such that a force (e.g., a predetermined radial force) exerted on wire 124 may cause wire 124 to constrict or otherwise reduce in size such that a proximal end of wire 124 may begin to move back up (i.e., proximally) or otherwise out of wound cavity 12 and/or esophagus 10. In some embodiments, a physician, or other user, may alternatively pull on a proximal end of wire 124 and/or an inner shaft or inner tube 128 (FIG. 2) to manually remove a portion of medical device 120 from wound cavity 12, for example, as wound cavity 12 decreases in size. The curved or helical shape of porous body 122 and wire 124 may also help to allow for medical device 120 to uncoil or straighten as medical device 120 is gradually removed by the user, for example, within outer tube 126. The change in size and/or configuration may help to allow medical device 120 to decrease in size simultaneously with wound cavity 12, such that medical device 120 does not need to be repositioned or removed and replaced with a smaller medical device 120 (e.g., a smaller porous body 122 and wire 124) as wound cavity 12 decreases in size.

In some examples, when porous body 122 is positioned over distal portion 124A of wire 124, an entirety of the exterior surface of distal portion 124A of wire 124 may be covered or surrounded by porous body 122. In other examples, porous body 122 may only cover one or more portion(s) of wire 124 (e.g., distal portion 124A of wire 124). For example, porous body 122 may include multiple pieces or may have a shape and configuration that leaves areas of wire 124 exposed. In some of these aspects, as shown in FIG. 2, porous body 122 may extend distal to distalmost end of distal portion 124A of wire 124, for example, to help form a soft, blunt, or otherwise atraumatic distal end of medical device 120.

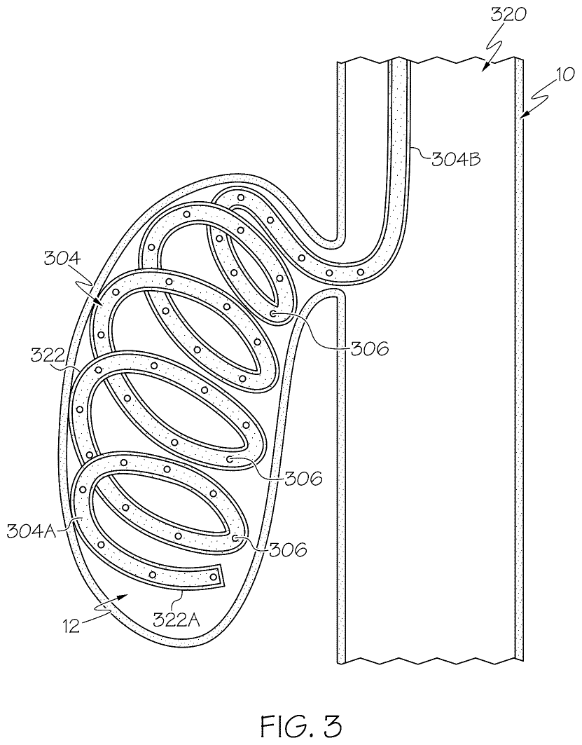

FIG. 3 illustrates another medical device 320 that includes a porous body 322 positioned radially around a tube, for example, a vacuum tube 304. In some aspects, vacuum tube 304 may be formed from a nasogastric tube. In some embodiments, vacuum tube 304 may be cylindrical with a central lumen extending along a central longitudinal axis of vacuum tube 304. Vacuum tube 304 may have a length and/or a width configured to extend through a working channel of an endoscope or other medical device, and may be flexible. Vacuum tube 304 may be coupled to, and in communication with, a vacuum or negative pressure source (not shown) at a proximal portion 304B of vacuum tube 304. The vacuum source may supply negative pressure to vacuum tube 304.

As shown in FIG. 3, vacuum tube 304 may include a plurality of suction ports 306 along a length of vacuum tube 304, for example, along a distal portion of vacuum tube 304. Suction ports 306 may be evenly or unevenly spaced along a portion (e.g., distal portion 304A) of vacuum tube 304. Suction ports 306 may help maintain negative pressure within wound cavity 12. For example, suction ports 306 may help to apply negative pressure to wound cavity 12 supplied by a suction or negative pressure source (e.g., a vacuum source), for example, coupled to a handle of medical device 320. Suction ports 306 may apply negative pressure to draw fluid through porous body 322, for example, through distal portion 322A of porous body 322. Accordingly, suction ports 306 may help to ensure that drainage of fluid is maintained as wound cavity 12 decreases in size throughout the healing process. In some aspects, one or more of suction ports 306 may also be used to deliver fluid (e.g., saline, an antibiotic fluid, etc.) to wound cavity 12, for example, to aid in the flushing and/or otherwise treat wound cavity 12.

In some embodiments, vacuum tube 304 may be formed from a polymer or any other suitable biocompatible material. Alternatively or additionally, in some embodiments, vacuum tube 304 may include a shape memory membrane, for example a Nitinol membrane, or vacuum tube 304 may be formed of a shape memory and/or heat-set material (e.g., Nitinol). In these aspects, vacuum tube 304 may be inserted into the working channel of an endoscope (not shown) in a straight, flat, or collapsed configuration prior to being deployed or otherwise positioned within a wound, for example, wound cavity 12. The straight configuration of vacuum tube 304 may aid in insertion of medical device 320 into a working channel of an endoscope, for example, the working channel of endoscope 110 (FIG. 1). Vacuum tube 304 may then be transitioned to an expanded configuration once deployed or otherwise positioned in wound cavity 12 (e.g., extended distally of the working channel opening). In some aspects, the expanded configuration of vacuum tube 304 may include a distal portion of vacuum tube 304 forming a curved or helical configuration. Additionally, in some embodiments, one or more of porous body 322 or vacuum tube 304 may have antiseptic properties, for example, to help prevent or inhibit infection and/or to help prolong the period for which porous body 322 may remain within wound cavity 12.

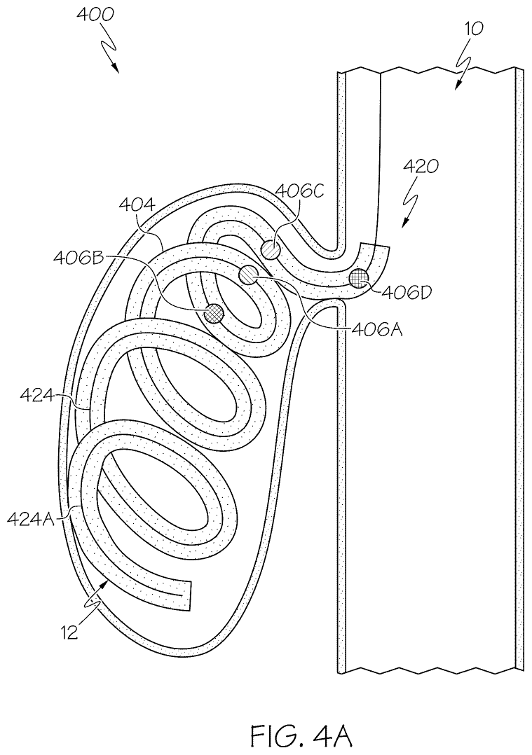

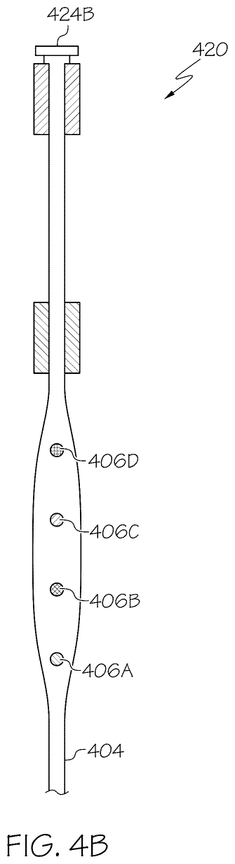

Additionally, any of the medical devices described herein may include one or more markers to aid the user in visualizing the medical device while deployed within the body. For example, as shown in FIGS. 4A and 4B, a medical device 420 (e.g., as part of a medical system 400) may include a plurality of markers 406A-D along a vacuum tube (not shown) or wire 424, for example, along a portion of the vacuum tube or wire 424 that may be adjacent to or near esophagus 10 when medical device 420 is positioned within wound cavity 12. At least a portion of wire 424 may include a porous body 404, as discussed above. At least a distal portion 424A of wire 424 may have a curved helical shape similar, as described above, and may also include a shape memory material which may allow wire 424 to be in a straight configuration (FIG. 4B) while being inserted into the working channel of endoscope 110 (FIG. 1), and then be transitioned to an expanded, curved or helical configuration (FIG. 4A) once deployed into wound cavity 12 or otherwise extended distal to working channel opening 116 of endoscope 110. In some aspects, markers 406A-406D may be spaced apart along one or more portions of the helical portion of wire 424. Moreover, although FIGS. 4A and 4B illustrate medical device 420 including wire 424, it is noted that, in some aspects, medical device 420 may include a vacuum tube, as discussed above, with the vacuum tube including one or more markers 406A-D.

As described above, during the healing process, distal portion 424A of wire 424 may be compressed as wound cavity 12 decreases in size (e.g., as wound cavity 12 heals). The reduction of size of wound cavity 12 may push or otherwise urge wire 424 out of wound cavity 12, for example, such that a proximal portion of wire 424 is urged into esophagus 10. Alternatively, a physician, or other user, may manually pull on a proximalmost end 424B of wire 424 (or on a tube coupled to wire 424) to manually remove a portion of medical device 420 (e.g., wire 424) from wound cavity 12. In either instance, it may be helpful for a physician, or other user, to be able to visualize how much of wire 424 remains within wound cavity 12 and/or how much of wire 424 has been moved into esophagus 10. Accordingly, in some embodiments, markers 406A-D may be included on wire 424, and may be easily visible using one or more fluoroscopy or imaging techniques. For example, in some embodiments, markers 406A-D may be color-coded and/or radiopaque. Therefore, a physician, or other user, may be able to determine the length of medical device 420 remaining within wound cavity 12 using fluoroscopy or one or more other imaging techniques at any point throughout the healing process. As a result, the user may easily be able to determine if a smaller or larger portion of medical device 420 should be removed from wound cavity 12, or otherwise monitor the healing process.

Additionally, in some embodiments, markers 406A-D may help to eliminate the need for a physician, or other user, to determine a size of wound cavity 12 prior to deploying or otherwise position medical device 420 into wound cavity 12. For example, markers 406A-D may be coded (e.g., color coded) such that a user may be able to visualize a certain code or marker on an excess length of medical device 420. For example, one or more of markers 406A-D may be different colors, sizes, shapes, patterns, etc., such that the user may be able to visualize and differentiate between markers 406A-D. Thus, the user may visualize a length or amount of medical device 420 that is within or external to wound cavity 12. Once an excess length of medical device 420 enters the esophageal lumen (e.g., esophagus 10), the user may visualize one or more of markers 406A-D in order to help ensure that an adequate length or portion of medical device 420 is positioned within wound cavity 12. Alternatively or additionally, markers 406A-D may help to ensure that porous body 404 at least partially seals (e.g., fully seals) wound cavity 12 from the esophageal lumen.

While principles of this disclosure are described herein with reference to illustrative examples for particular applications, it should be understood that the disclosure is not limited thereto. For example, the disclosure refers to EVAC as an exemplary procedure, and the GI tract as a typical lumen for the systems and methods of the disclosure. The systems, devices, and methods of the present disclosure, however, may be used in any suitable medical procedure in any lumen or cavity within the body, for example, to aid in drainage from a wound anywhere within the body. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical device, comprising:
a wire or tube; and
a porous body coupled a distal portion of the wire or tube,
wherein the wire or tube is configured to be transitioned from a first configuration to a second configuration,
wherein, in the first configuration, the wire or tube is in a flat or straight configuration, and
wherein, in the second configuration, the wire or tube is expanded to a curved or helical shape.

2. The medical device of claim 1, wherein at least a distal portion of the wire or tube includes or is formed of a shape memory material.

3. The medical device of claim 1, wherein the wire or tube is a vacuum tube configured to be coupled to a negative pressure source.

4. The medical device of claim 3, wherein the vacuum tube is cylindrical with a central lumen extending along a central longitudinal axis of the vacuum tube.

5. The medical device of claim 4, wherein the vacuum tube includes a plurality of ports disposed along a length of a distal portion of the vacuum tube, and wherein the plurality of ports are configured to apply negative pressure provided by the negative pressure source.

6. The medical device of claim 1, wherein, in the second configuration, the curved or helical shape of the wire or tube causes the porous body to have a helical shape.

7. The medical device of claim 1, wherein the porous body is a sponge.

8. The medical device of claim 1, wherein the porous body is cylindrical.

9. The medical device of claim 1, wherein the wire or tube is configured to be deployed into a wound cavity, and wherein a length of wire vacuum tube within the wound cavity is configured to be reduced as the wound cavity reduces in size.

10. The medical device of claim 1, wherein the wire or tube remains in the first configuration when exposed to a first temperature, and transitions to the second configuration upon being exposed to a second temperature.

11. The medical device of claim 10, wherein the second temperature is body temperature.

12. The medical device of claim 1, wherein the porous body covers only a portion of a distal end of the wire or tube.

13. The medical device of claim 1, wherein the porous body covers an entirety of a distal end of the wire or tube.

14. The medical device of claim 1, further comprising a plurality of markers disposed along a length of a portion of the wire or tube.

15. The medical device of claim 14, wherein the plurality of markers are radiopaque.

16. A medical device, comprising:
a vacuum tube configured to be inserted into a working channel of an endoscope, and further configured to be coupled to a negative pressure source; and
a porous body coupled a distal portion of the vacuum tube,
wherein the vacuum tube is configured to be transitioned from a first configuration to a second configuration,
wherein, in the first configuration, the vacuum tube is flat or straight, and
wherein, in the second configuration, the vacuum tube is expanded and distal portions of the vacuum tube and the porous body have a helical shape.

17. The medical device of claim 16, wherein the vacuum tube remains in the first configuration when exposed to a first temperature, and transitions to the second configuration upon being exposed to a second temperature.

18. The medical device of claim 17, further comprising a plurality of markers disposed along a length of the vacuum tube, wherein the plurality of markers are radiopaque and/or color coded.

19. A medical device, comprising:
a wire or tube, wherein at least a distal portion of the wire or tube includes or is formed of a shape memory material, and wherein the wire or tube includes a plurality of radiopaque markers; and
a porous body coupled to the distal portion of the wire or tube,
wherein the wire or tube is configured to be transitioned from a first configuration to a second configuration,
wherein, in the first configuration, the wire or tube is in a flat or straight configuration, and
wherein, in the second configuration, the wire or tube is expanded to a curved or helical shape.

20. The medical device of claim 19, wherein the wire or tube is a vacuum tube configured to be coupled to a negative pressure source,
wherein the vacuum tube includes a central lumen extending along a central longitudinal axis of the vacuum tube, and
wherein the vacuum tube includes a plurality of ports disposed along a length of a distal portion of the vacuum tube, and wherein the plurality of ports are configured to apply negative pressure provided by the negative pressure source.
